(19) United States
(12) Patent Application Publication
(10) Pub. No.: US 2004/0014804 A1
(43) Pub. Date: Jan. 22, 2004

(54) Methods for producing amino-substituted chromanes

(76) Inventors: Luc Antoine, Landen (BE); Pascal Bouquel, Montmedy (FR); Alfio Borghese, Baisy-Thy (BE); Hugo Gorissen, Grez-Doiceau (BE); Michael Martinelli, Drive Zionsville, IN (US); Alain Merschaert, Louche (BE); Gerd Ruhter, Hamburg (DE); Carine Rypens, Gorges (FR); Robert Scarborough, Half Moon Bay, CA (US); Theo Schotten, Vierhofen (DE); Jean-Pierre Van Hoeck, Chastre (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/296,782

(22) PCT Filed: Jun. 1, 2001
(86) PCT No.: PCT/US01/18014

(51) Int. Cl. A61K 31/353; C07D 311/76

(52) U.S. Cl. 514/456; 549/404

(57) ABSTRACT

Disclosed are processes for producing benzopyran compounds, reduced 2-(chroman-2-yl) acetic acid compounds and 2-(6-aminochroman-2-yl)acetic acid esters which are intermediates for producing platelet aggregation inhibitors and/or are themselves potent platelet aggregation inhibitors.

### FIELD OF THE INVENTION

This invention relates to novel processes for producing chromane compounds, preferably chroman-2-yl acetic acid compounds and amino substituted chroman-2-yl acetic acid esters which are intermediates for producing platelet aggregation inhibitors and/or are themselves potent platelet aggregation inhibitors. Further the invention relates to processes for resolving chiral intermediates or final products to provide desired enantiomers.

### BACKGROUND OF THE INVENTION

One process for making benzopyrans from coumarin derivatives is described in U.S. Pat. No. 5,731,324 at pages 101-103. However, that process involves chromatography as a purification step, which does not scale well commercially. The unprotected amino derivative bicyclic compound is shown on page 147

### SUMMARY OF THE INVENTION

In accordance with one preferred embodiment, there is provided a process for making a compound according to the formula

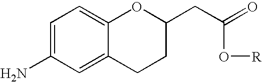

wherein R is H or an alkyl group. The process comprises the following reactions (a) through (d):

(a) reacting a 6-nitro coumarin compound with at least one hydrogenation agent to hydrogenate the 6-nitro group to a 6-amino group and concurrently or in a further step hydrogenate the 3-4 alkene bond on the lactone ring to produce a R¹-6-amino-2-chromanone compound as follows:

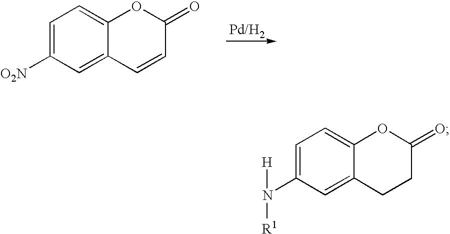

wherein the R¹ group is a hydrogen atom or a removable amino protecting selected from the group consisting of BOC, t-butoxycarbonyl, and the like,

(b) reducing the 2 position carbonyl group of the protected 6-amino-2-chromanone from (a) with a suitable carbonyl reducing compound such as DIBAL-H in a suitable organic solvent to form a protected 6-amino-2-hydroxychromane as follows:

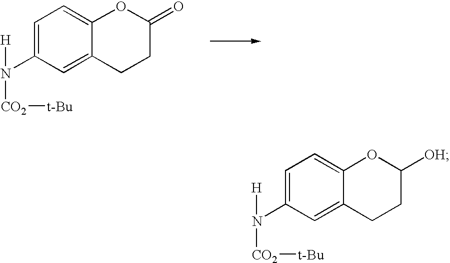

(c) condensing the hydroxychromane compound of (b) with a triphenylphosphorane compound in the presence of a suitable base, preferably at a temperature of about 50-100° C., to afford the acetate compound as follows:

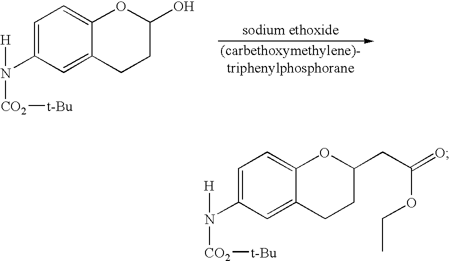

(d) removing the protecting group from the 6-amino groupsuch as by using trifluoroacetic acid at a temperature of about 40-80° C., as follows:

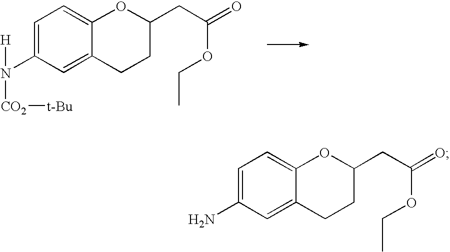

Optionally, one may also form the free acetic acid side chain or perform a transesterification process step to provide a compound of the formula:

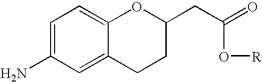

wherein R is H or an alkyl group.

In accordance with another preferred embodiment, the process is used to prepare a compound according to the formula:

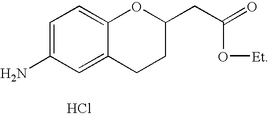

### DETAILED DESCRIPTION OF THE PREFERRED EMBODIMENTS

Because of the disadvantages of the aforementioned process, there is a need for improved processes for producing compounds that are useful as intermediates in processes for producing platelet aggregation inhibitors. There is a particular need for improved processes for making compounds having the phenyl ring of the benzopyrans substituted by an amino group or a protected amino group. Such intermediates are useful for coupling with a carbonyl group to produce a carboxamide link and result in compounds that are useful platelet aggregation inhibitors or intermediates for forming platelet aggregation inhibitors. Also needed is a process to produce relatively inexpensively large quantities of chromone intermediates that are useful for being resolved by conventional processes to produce benzopyran or chromane derivatives wherein the chiral center at the two position of the saturated pyran ring portion of the bicyclic ring structure can be resolved into racemic mixtures (RIS) that are enriched with one of the R or S enantiomers or to produce substantially pure compositions of a single enantiomer (R or S enantiomer). Due to inherent losses of up to 50% or more of the starting materials (assuming a 50/50 R/S racemate) during enantiomeric resolution, there is a need for a process which is efficient enough to be scaled to an industrial level for inexpensively producing large quantities of a desired intermediate compound or large quantities of final chroman-2-yl acetic acid ester compounds that are useful in the anticoagulant field.

Accordingly, there continues to be a need for a process that is adaptable to commercially scaleable production of such benzopyrans. One or more of the foregoing needs may be met using the processes described herein and the compounds and intermediates made thereby.

In one aspect, the processes herein relate to producing chromane compounds, preferably chroman-2-yl acetic acid compounds and amino substituted chroman-2-yl acetic acid esters which are intermediates for producing therapeutic agents, or are themselves therapeutic agents, for disease states in mammals that have disorders caused by or impacted by platelet dependent narrowing of the blood supply.

In particular, one preferred embodiment provides a process that utilizes a 6-nitro coumarin compound (available from Aldrich) and in the first step reduces the 6-nitro group to a substituted amino group and reduces the 3-4 alkene bond to produce a 6-(tert-butoxy-carbonylamino)-2-chromanone compound as follows:

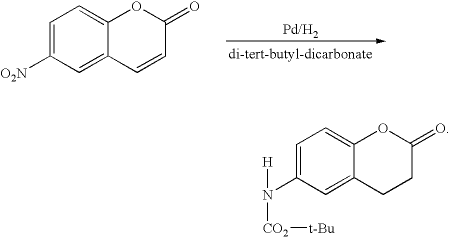

Ideally, the reduction of the nitro group and the cyclic 3-4 alkene bond are conducted in a single step. For example, palladium on carbon or the like may be used in a solvent such as THF to reduce both the nitro group and the cyclic alkene bond. More preferably, di-tert-butyl-dicarbonate is added to reaction mixture along with the THF and results in protecting the amino group as it is formed. A lower hydrogen pressure and temperature may be used initially followed by increasing of the hydrogen pressure and the temperature to reduce the cyclic alkene bond.

The carbonyl group (2-oxo group) of the protected 2-chromanone from the above step can be reduced to a 2-hydroxychromane by utilizing a DIBAL-H reduction process or the like as follows:

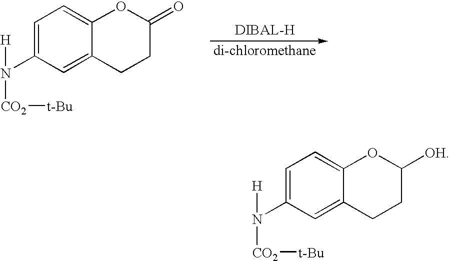

The 2-hydroxy chromane is then condensed with a (carbethoxymethylene)-triphenylphosphorane compound in the presence of a base such as sodium ethoxide in an acceptable solvent such as toluene at about 50-100° C., preferably about 65-90° C., and more preferably about 80° C. to afford the acetate. For example, as illustrated below:

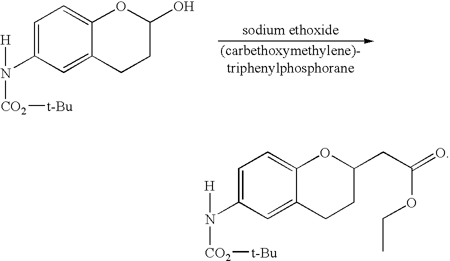

The protecting group on the amine group can then optionally be removed with an acceptable acid such as trifluoroacetic acid at about 40-80° C., preferably 50-70° C., and more preferably about 60° C. to yield the free amine as follows:

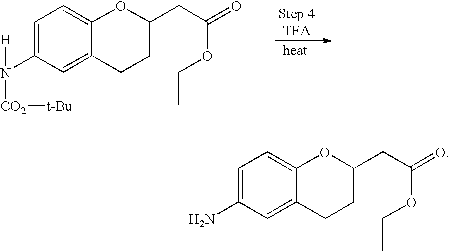

While an ethyl group was used to form the ester of the acetic acid side chain, the ethyl group can be replaced by H or another esterifying group selected from lower alkyl, lower alkenyl, lower alkynyl, phenyl, cinnamyl or other ester groups.

In either event, the protected amine benzopyran compound or the free amine benzopyran compound can be coupled to a cyanobenzoyl chloride group as described on pages 147 and 148 of U.S. Pat. No. 5,731,324, for example. The ester group of the acetic acid side chain can be optionally changed, before or after the coupling step.

Further, the above process can be modified to produce a formyl, propyl or butyl side chain or the like, by utilizing a different triphenylphosphorane starting material.

Uses of Compounds

As mentioned above, the compounds disclosed herein find utility as intermediates for producing therapeutic agents or as therapeutic agents for disease states in mammals which have disorders that are due to platelet dependent narrowing of the blood supply, such as atherosclerosis and arteriosclerosis, acute myocardial infarction, chronic stable angina, unstable angina, transient ischemic attacks and strokes, peripheral vascular disease, arterial thrombosis, preeclampsia, embolism, restenosis following angioplasty, carotid endarterectomy, anastomosis of vascular grafts, and etc. These conditions represent a variety of disorders thought to be initiated by platelet activation on vessel walls.

Platelet adhesion and aggregation is believed to be an important part of thrombus formation. This activity is mediated by a number of platelet adhesive glycoproteins. The binding sites for fibrinogen, fibronectin and other clotting factors have been located on the platelet membrane glycoprotein complex IIb/IIIa. When a platelet is activated by an agonist such as thrombin the GP IIb/IIIa binding site becomes available to fibrinogen, eventually resulting in platelet aggregation and clot formation. Thus, intermediate compounds for producing compounds that effective in the inhibition of platelet aggregation and reduction of the incidence of clot formation are useful intermediate compounds.

The compounds produced according to the methods disclosed herein may used as intermediates for producing therapeutic compounds or as compounds that may be administered in combination or in concert with other therapeutic or diagnostic agents. In certain preferred embodiments, the compounds produced by the intermediates according to the disclosure herein may be co-administered along with other compounds typically prescribed for these conditions according to generally accepted medical practice such as anticoagulant agents, thrombolytic agents, or other antithrombotics, including platelet aggregation inhibitors, tissue plasminogen activators, urokinase, prourokinase, streptokinase, heparin, aspirin, or warfarin. The compounds produced from the intermediates according to preferred embodiments may act in a synergistic fashion to prevent reocclusion following a successful thrombolytic therapy and/or reduce the time to reperfusion. Such compounds may also allow for reduced doses of the thrombolytic agents to be used and therefore minimize potential hemorrhagic side-effects. Such compounds can be utilized in vivo, ordinarily in mammals such as primates, (e.g. humans), sheep, horses, cattle, pigs, dogs, cats, rats and mice, or in vitro.

The starting materials used in above processes are commercially available from chemical vendors such as Aldrich, Sigma, Nova Biochemicals, Bachem Biosciences, and the like, or may be readily synthesized by known procedures, for example, by using procedures such as indicated above.

Reactions are carried out in standard laboratory glassware and reaction vessels under reaction conditions of standard temperature and pressure, except where otherwise indicated, or is well-known in literature available in the art. Further, the above procedures of the processes described herein may be carried out on a commercial scale by utilizing reactors and standard scale-up equipment available in the art for producing large amounts of compounds in the commercial environment. Such equipment and scale-up procedures are well-known to the ordinary practitioner in the field of commercial chemical production.

During the synthesis of these compounds, amino or acid functional groups may be protected by blocking groups to prevent undesired reactions with the amino group during certain procedures. Examples of suitable blocking groups are well known in the art. Further, removal of amino or acid blocking groups by procedures such as acidification or hydrogenation are well-known in the art.

Enantiomeric Resolution and Acid Salt Formation

As is clear from the above formulae and the discussion above, the two-position acid ester group is attached to a chiral carbon which may optionally be resolved to produce a racemic mixture enriched in either the R or S enantiomers or completely resolved into a substantially pure composition of one of the enantiomers. Conventional processes may be utilized to resolve the enantiomers.

Compositions and Formulations

The compounds according to preferred embodiments may be isolated as the free acid or base or converted to salts of various inorganic and organic acids and bases. Such salts are within the scope of this disclosure and are presently contemplated. Non-toxic and physiologically compatible salts are particularly useful although other less desirable salts may have use in the processes of isolation and purification.

A number of methods are useful for the preparation of the salts described above and are known to those skilled in the art. For example, reaction of the free acid or free base form of a compound of the structures recited above with one or more molar equivalents of the desired acid or base in a solvent or solvent mixture in which the salt is insoluble, or in a solvent like water after which the solvent is removed by evaporation, distillation or freeze drying. Alternatively, the free acid or base form of the product may be passed over an ion exchange resin to form the desired salt or one salt form of the product may be converted to another using the same general process.

Without further description, it is believed that one of ordinary skill in the art can, using the preceding description and the following illustrative examples, make and utilize the disclosed and claimed compounds and practice the disclosed and claimed methods. The following working examples therefore, specifically point out preferred embodiments, and are not to be construed as limiting in any way the remainder of the disclosure.

### EXAMPLES

### Example 1

### Production of 6-(t-butoxy-carbonylamino)-2-chromanone

Ten percent palladium on carbon (2.89 Kg of wet material; about 50% moisture) and 3 angstrom molecular sieves (13.73 Kg) were placed in a pressure reactor. The reactor was purged 3 times with nitrogen. Tetrahydrofuran (53 Kg) was added under vacuum while maintaining the reaction medium at room temperature (controlling the exothermic reaction). 6.85 Kg of 6-nitrocoumarin (35.84 mole) and 8.6 Kg of di-tert-butyl-dicarbonate (39.40 mole, about 1.1 equivalents with respect to the 6-nitrocoumarin) were added to the stirred solution. The reactor was purged 3 times with nitrogen and then 3 times with hydrogen. The pressure of hydrogen was maintained at about 2 bar G until the end of exothermicity (20° C.≦T≦45° C.).

The solution was then heated to about 50° C. and the pressure of hydrogen was increased to about 4 bar G for about 5 hours. When the reaction was essentially complete (the area ratio by HPLC between the amount of 6-nitro-coumarin starting material and the amount of 6-(t-butoxy-carbonylamino)-2-chromanone product was not more than about 3%), the mixture was cooled to room temperature. The suspension was filtered and the cake was washed with 30 Kg of tetrahydrofuran. The filtrate was distilled under reduced pressure (T≦60° C.) by adding about 23 Kg of toluene and distilling off about 8 Kg of the solvent. The medium was then heated to reflux until complete dissolution. The mixture was slowly cooled down and stirred at room temperature for about 2 hours or more.

The crystals were filtered, washed twice with 10 Kg of toluene and dried for about 16 hours under reduced pressure (20° C.≦T≦45° C.) to yield 7.89 Kg of 6-(t-butoxycarbonylamino)-2-chromanone (29.97 mole). Yield about 83.6%.

¹H-NMR (250 MHz, CDCl₃) 7.39 (s, 1H), 7.84 (dd, J=10.3 Hz, 2.5 Hz, 1H), 6.98 (s, 1H), 6.87 (d, J=10 Hz, 1H), 2.90 (t, J=7.5 Hz, 2H), 2.70 (td, J=7.5 Hz, 2.5 Hz, 2H), 1.46 (s, 9H)

¹³C-NMR (62.9 MHz, CDCl₃) 168.6, 152.9, 147.2, 134.8, 122.9, 116.2, 118.2, 118.0, 116.8, 28.9, 28.1, 23.6.

### Example 2

### Production of 6-(t-butoxycarbonylamino)-2-hydroxychromane

About 12.01 Kg of 6-(t-butoxycarbonylamino)-2-chromanone (produced as set forth in Example 1, above) was added to 159 Kg of dichloromethane while stirring at room temperature. The solution was then cooled to about −55° C. and 36.90 Kg of diisobutylaluminum hydride (DIBAL-H, 25% w/w in toluene; 65.34 mole) was slowly added over about 1 hour. The rate of addition was adapted in order to keep the temperature between −50° C. and −65° C. (exothermic). The reaction mixture was stirred for about 15 minutes after the DIBAL-H had been added. Methyl alcohol (11.4 Kg) was then slowly added (T≦−50° C.), and upon completion of the addition, the reaction mixture was slowly warmed up to about −10° C. When this temperature was obtained, 12 Kg of celite and 15.6 Kg of water were added. The mixture was further warmed-up to 20° C. and maintained under good agitation at that temperature for at least 30 minutes. The suspension was filtered and the cake was washed 3 times with 76.85 Kg of dichloromethane. The filtrate and washes were combined and the solvent was distilled off under reduced pressure (T≦50° C.) until the minimum stirrable volume was reached (about 40 L).

Toluene (36.3 Kg) was added to the reactor and the solvents were distilled until minimum stirrable volume was achieved. This operation was repeated 3 times. Finally, 52 Kg of toluene was added and distilled under reduced pressure until the residual volume was about 132 L. The reaction mixture was then cooled to room temperature to yield 6-(t-butoxycarbonylamino)-2-hydroxychromane in a toluenic solution.

¹H-NMR (400 MHz, CDCl₃) 7.17 (d(br), J=2.5 Hz, 1H), 6.96 (dd, J=8.9 Hz, 2.5 Hz, 1H), 6.72 (d, J=8.9 Hz, 1H),6.36 (s, 1H), 5.57 (t, J=3.2 Hz, 1H), 2.93 (ddd, J=16.5 Hz, 10.4 Hz, 5.2 Hz, 1H), 2.6 (s, 1H), 1.97 (m, 2H), 1.50 (s, 9H).

¹³C-NMR (100 MHz, CDCl₃) 153.3, 148.1, 131.3, 129.1, 128.2, 125.3, 122.4, 188.8, 117.1, 92.1, 80.3, 28.4, 27.0, 20.5.

### Example 3

### Production of Ethyl [6-(t-butoxycarbonylamino)chroman-2-yl]acetate

To the toluenic solution of Example 2, above, was added 17.53 Kg of (carbethoxymethylene)triphenylphosphorane (50.32 mole, corresponding to 1:1 equivalents of the amount of 6-(t-butoxycarbonylamino)-2-chromanone used as the initial starting material in Example 2) and 16 g of sodium ethoxide. The reaction mixture was then heated to about 80° C. and stirred at 80° C. for at least about 2 hours. The evolution of the reaction was then checked by TLC and HPLC. An additional amount of sodium ethoxide (48 g) was added and the mixture was maintained at about 80° C. for about 24 hours with stirring. After cooling the reaction mixture to room temperature, 57.11 Kg of silica gel and 91 Kg of toluene were added to the reaction medium which was stirred for at least 1 hour at about 20° C.

The silica gel was filtered and washed twice with 122 Kg of toluene. The filtrate and the washes were pooled and the solvents were distilled off under reduced pressure (T≦50° C.) until the residue solution had a volume of about 100 L. The residue was cooled down to about 25° C. to result in a 100 L toluenic solution of ethyl [6-(t-butoxy carbonylamino) chroman-2-yl] acetate.

### Example 4

### Production of Ethyl [6-(amino)chroman-2-yl]acetate

To the toluenic solution of Example 3, above, was added 25 Kg of trifluoro acetic acid (219.26 mole). The solution was then heated up to about 60° C. for at least one hour. The solution cooled down to about 40° C. and the solvents were distilled off (T≦50° C.) under reduced pressure until the volume of the residue was about 100 L.

The mixture was cooled down to room temperature and 10% (w/w) aqueous sodium hydrogen carbonate was slowly added until the pH was above 7 (112 Kg were added). The solution was stirred for at least 15 minutes. The organic and aqueous layers were separated and the aqueous layer was extracted twice with 23.4 kg of toluene. The combined organic layers were distilled off under reduced pressure (T≦50° C.) until the residue was about 80 L (toluenic solution of ethyl [6-(amino)chroman-2-yl]acetate.

¹H-NMR (400 MHz, CDCl₃) 6.61 (d, J=8.9 Hz, 1H), 6.46 (dd, J=8.9 Hz, 2.5 Hz, 1H), 6.40 (d, J=2.5 Hz, 1H), 4.37 (qd, J=7.5 Hz, 1.2 Hz, 1H), 4.18 (q, J=7.2 Hz, 2H), 3.22 (s, 2H), 2.81 (ddd, J=16.5 Hz, 5.2 Hz, 4.1 Hz, 1H), 2.58 (dd, J=15.4 Hz, 7.4 Hz, 1H), 2.02 (dm, J=13.5 Hz, 1H), 1.75 (m, 1H), 1.07 (t, J=7.2 Hz, 3H).

¹³C-NMR (100 MHz, CDCl₃) 107.9, 147.5, 139.4, 122.1, 117.3, 115.9, 115.0, 72.1, 60.6, 40.6, 27.3, 24.5, 14.2.

### Example 5

### Optional Production of Ethyl [6-(amino)chroman-2-yl]acetate Hydrochloride

The 80 L of the toluenic solution of Example 4, above, was cooled to 19° C. and 9.67 L of 6.2 M hydrochloric acid in ethyl alcohol was slowly added in order to maintain the temperature between 10 and 20° C. The crystals were maturated under stirring for at least 16 hours at the same temperature, filtered and washed with 40 L of toluene. The product was dried for at least 16 hours under reduced pressure while maintaining the temperature between 45 and 50° C. to afford about 8.50 Kg (31.34 mole) of ethyl [6-amino-2-chroman-2-yl]acetate hydrochloride. Yield about 68.7% for production steps from 6-(t-butoxycarbonylamino)-2-chromanone to ethyl [6-amino-2-chroman-2-yl]acetate hydrochloride salt.

¹H-NMR (250 MHz, DMSO-d6),9.97 (s,(br), 3H), 7.05 (m, 2H, 6.80 (m, 1H), 4.42 (qd, J=7.5 Hz, 1.2 Hz, 1H), 4.14 (q, J=6.7 Hz, 2H), 2.88 (ddd, J=16.5 Hz, 10.4 Hz, 5.2 Hz, 1H), 2.80 (dd, j=11.3 Hz, 6.7 Hz, 1H), 1.70 (m, 4H), 1.22 (t, J=6.7 Hz, 3H)

¹³C-NMR (62.9 MHz, DMSO-d6), 170.2, 153.4, 123.9, 123.1, 121.9, 117.3, 72.6, 60.1, 39.7, 25.9, 23.7, 14.1.

### Example 6

### Production of Toluenic Solution of Ethyl [6-(amino)chroman-2-yl]acetate from the 6-amino Hydrochloride Salt

At room temperature the 8.5 Kg of ethyl [6-aminochroman-2-yl]acetate hydrochloride salt of Example 5 was added to 80 L of toluene with stirring and 10% (w/w) aqueous sodium hydrogen carbonate was slowly added until the pH was above 7 and all of the salt had dissolved in the reaction mixture. The solution was stirred for about 15 minutes. The organic and aqueous layers were separated. The aqueous layer was extracted twice with 23.4 Kg of toluene. The combined organic layers were distilled off under reduced pressure (T≦50° C.) until the residue was about 80 L to yield a toluenic solution of ethyl [6-(amino)chroman-2-yl]acetate.

¹H-NMR (400 MHz, CDCl₃) 6.61 (d, J=8.9 Hz, 1H), 6.46 (dd, J=8.9 Hz, 2.5 Hz, 1H), 6.40 (d, J=2.5 Hz, 1H), 4.37 (qd, J=7.5 Hz, 1.2 Hz, 1H), 4.18 (q, J=7.2 Hz, 2H), 3.22 (s, 2H), 2.81 (ddd, J=16.5 Hz, 5.2 Hz, 4.1 Hz, 1H), 2.58 (dd, J=15.4 Hz, 7.4 Hz, 1H), 2.02 (dm, J=13.5 Hz, 1H), 1.75 (m, 1H), 1.07 (t, J=7.2 Hz, 3H).

¹³C-NMR (100 MHz, CDCl₃) 107.9, 147.5, 139.4, 122.1, 117.3, 115.9, 115.0, 72.1, 60.6, 40.6, 27.3, 24.5, 14.2.

### Example 7

### Production of 6-(t-butoxy-carbonylamino)-2-chromanone

Into an 8 L hydrogenation reactor, equipped with an agitator, temperature probes, heating and cooling systems, was charged under nitrogen, 125 g of 10% Palladium on carbon, 300 g of 3 angstrom powdered molecular sieves, 1050 g (5.5 moles) of 6-nitro-chrom-3-en-2-one (6-nitro coumarin), 1318 g (6.1 moles) of di-tert-butyldicarbonate, and 4.0 L of anhydrous tetrahydrofuran. The reactor was purged 5 times with nitrogen and then 5 times with hydrogen. The pressure of hydrogen was maintained at 30 psi until the end of exothermicity (30° C.≦T≦50° C.).

The reaction mixture was then heated to about 50° C. and the pressure of hydrogen was increased to about 60 psi. During the reaction process TLC was used to monitor the progress of the reaction, and the reaction was complete in about 15 hours. Upon completion, the reaction mixture was cooled to room temperature and purged 3 times with nitrogen before it was discharged from the reactor. The catalyst and molecular sieves were removed by filtration through a layer of celite and the filtration cake was washed with 1.0 L of tetrahydrofuran.

The filtrate was concentrated to dryness at 50° C. under reduced pressure to give off-white crude product. The crude product was recrystallized in 3.6 L of toluene. The first crop afforded 1300 g of the product 6-(t-butoxycarbonylamino)-2-chromanone and the mother liquor was concentrated to give an additional 47 g of the product (total yield 93.1%).

¹H-NMR (250 MHz, CDCl₃) 7.39 (s, 1H), 7.84 (dd, J=10.3 Hz, 2.5 Hz, 1H), 6.98 (s, 1H), 6.87 (d, J=10 Hz, 1H), 2.90 (t, J=7.5 Hz, 2H), 2.70 (td, J=7.5 Hz, 2.5 Hz, 2H), 1.46 (s, 9H)

¹³C-NMR (62.9 MHz, CDCl₃) 168.6, 152.9, 147.2, 134.8, 122.9, 116.2, 118.2, 118.0, 116.8, 28.9, 28.1, 23.6.

### Example 8

### Production of 6(t-butoxy-carbonylamino)-2-hydroxychromane

Into a 22 L three-neck round bottom flask equipped with an overhead stirrer and an addition funnel was charged, under nitrogen, 7 L of anhydrous methylene chloride and 800 g of 6-(t-butoxycarbonylamino)-2-chromanone (produced as set forth in Example 1, above). The solution was then cooled to about −55° C. and 2.66L (1.58 M solution in toluene, 4.2 moles) of diisobutylaluminum hydride (DIBAL-H) was slowly added over about 1½ hours through the addition funnel with good stirring. The rate of addition was adapted in order to keep the temperature between −50° C. and −65° C. (exothermic). The reaction mixture was stirred for 1 hour after the DIBAL-H was added. The 870 mL of methanol was then slowly added (T≦−50° C.). The reaction mixture was warmed up, and at about −20° C., and 790 g of celite and 970 mL of water were added. The mixture was further warmed-up to room temperature and maintained under good agitation at that temperature for about 40 minutes. The suspension was filtered and the cake was washed with 10.0 L of methylene chloride in three washings. The filtrate and the washings were combined and the solvent was removed under reduced pressure (T≦50° C.). The residue was transferred with 8.0 L of toluene to a 22 L three-necked round bottomed flask. Azeotropic removal of water was done by distilling 6 L of toluene under reduced pressure at about 50° C. The volume of remaining solution was adjusted to a volume of about 8 L by adding anhydrous toluene to yield 6-(t-butoxycarbonylamino)-2-chromanone in a toluenic solution.

¹H-NMR (400 MHz, CDCl₃) 7.17 (d(br), J=2.5 Hz, 1H), 6.96 (dd, J=8.9 Hz, 2.5 Hz, 1H), 6.72 (d, J=8.9 Hz, 1H),6.36 (s, 1H), 5.57 (t, J=3.2 Hz, 1H), 2.93 (ddd, J=16.5 Hz, 10.4 Hz, 5.2 Hz, 1H), 2.6 (s, 1H), 1.97 (m, 2H), 1.50 (s, 9H).

¹³C-NMR (100 MHz, CDCl₃) 153.3, 148.1, 131.3, 129.1, 128.2, 125.3, 122.4, 188.8, 117.1, 92.1, 80.3, 28.4, 27.0, 20.5.

### Example 9

### Production of Ethyl [6(t-butoxycarbonylamino)chroman-2-yl]acetate

To the toluenic solution of Example 8, above, was added 1350 g of (carbethoxymethylene)triphenylphosphorane (95%) and 2.1 g of sodium ethoxide. The reaction mixture was then heated to about 80° C. and stirred at 80° C. for 2 hours. An additional 3.2 g of sodium ethoxide was added and the temperature maintained with stirring for 18 hours. TLC analysis was used to monitor the evolution of the reaction from time to time. The reaction mixture was stirred at 80° C. for an additional 3 hours after the addition. After cooling the reaction mixture to room temperature, 3750 g of silica gel and 70 L of toluene were added to the reaction medium which was stirred for 2 hours before filtration.

After filtration, the silica gel was washed with 2×12 L of toluene (total of 24 L of toluene). The filtrate and the washes were combined and the solvent was distilled off under reduced pressure (T # 60° C.) until the residue solution had a volume of about 7 L. The residue was cooled down to about 25° C. to result in an 8 L toluenic solution of ethyl [6-(t-butoxycarbonylamino)chroman-2-yl]acetate.

### Example 10

### Production of Ethyl [6-(amino)chroman-2-yl]acetate

To the toluenic solution (8 L) of Example 9, above, was added 1480 g (13.1 moles) of trifluoroacetic acid. The solution was then heated up to about 60° C. for two hours. The solution was cooled down to about 40° C. and about 2 L of the toluene solvent was removed by distillation under reduced pressure (at T≦60° C.).

The mixture was cooled down to room temperature and about 10 L of 10% (w/w) aqueous sodium hydrogen carbonate was slowly added until the pH was above 7. The solution was stirred for 20 minutes. The organic and aqueous layers were separated and the aqueous layer was extracted with 3 L of toluene. The combined organic layers were washed with 3 L of brine, dried over sodium sulfate, and filtered. The toluene solvent was removed by distillation under reduced pressure (at T≦60° C.) until the residue was about 6 L (toluenic solution of ethyl [6-(amino)chroman-2-yl]acetate).

¹H-NMR (400 MHz, CDCl₃) 6.61 (d, J=8.9 Hz, 1H), 6.46 (dd, J=8.9 Hz, 2.5 Hz, 1H), 6.40 (d, J=2.5 Hz, 1H), 4.37 (qd, J=7.5 Hz, 1.2 Hz, 1H), 4.18 (q, J=7.2 Hz, 2H), 3.22 (s, 2H), 2.81 (ddd, J=16.5 Hz, 5.2 Hz, 4.1 Hz, 1H), 2.58 (dd, J=15.4 Hz, 7.4 Hz, 1H), 2.02 (dm, J=13.5 Hz, 1H), 1.75 (m, 1H), 1.07 (t, J=7.2 Hz, 3H).

¹³C-NMR (100 MHz, CDCl₃) 107.9, 147.5, 139.4, 122.1, 117.3, 115.9, 115.0, 72.1, 60.6, 40.6, 27.3, 24.5, 14.2.

### Example 11

### Optional Production of Ethyl [6-(amino)chroman-2-yl]acetate Hydrochloride

The 6 L of the toluenic solution of Example 11, above, was cooled to 19° C. and 0.75 L of 6.2 M hydrochloric acid in ethyl alcohol was slowly added in order to maintain the temperature between 10 and 20° C. The crystals were maturated under stirring for at least 16 hours at the same temperature, filtered and washed with 3 L of toluene. The product was dried for at least 16 hours under reduced pressure while maintaining the temperature between 45 and 50° C. to afford about 1.07 Kg (3.97 mole) of ethyl [6-aminochroman-2-yl]acetate hydrochloride salt. Yield about 77.5% for production steps from 6-(t-butoxycarbonylamino)-2-chromanone to the ethyl [6-aminochroman-2-yl]acetate hydrochloride salt, and an overall yield of about 72% for production from the 6-nitro-coumarin to the ethyl [6-aminochroman-2-yl]acetate hydrochloride salt.

¹H-NMR (250 MHz, DMSO-d6),9.97 (s,(br), 3H), 7.05 (m, 2H, 6.80 (m, 1H), 4.42 (qd, J=7.5 Hz, 1.2 Hz, 1H), 4.14 (q, J=6.7 Hz, 2H), 2.88 (ddd, J=16.5 Hz, 10.4 Hz, 5.2 Hz, 1H), 2.80 (dd, j=11.3 Hz, 6.7 Hz, 1H), 1.70 (m, 4H), 1.22 (t, J=6.7 Hz, 3H)

¹³C-NMR (62.9 MHz, DMSO-d6), 170.2, 153.4, 123.9, 123.1, 121.9, 117.3, 72.6, 60.1, 39.7, 25.9, 23.7, 14.1.

### Example 12

### Production of Toluenic Solution of Ethyl [6-aminochroman-2-yl]acetate from the 6-amino Hydrochloride Salt

At room temperature the 1.07 Kg of ethyl [6-aminochroman-2-yl]acetate hydrochloride salt of Example 11 was added to 6 L of toluene with stirring and 10% (w/w) aqueous sodium hydrogen carbonate was slowly added until the pH was above 7 and all of the salt had dissolved in the reaction mixture. The solution was stirred for about 15 minutes. The organic and aqueous layers were separated. The aqueous layer was extracted twice with 2 L of toluene. The combined organic layers were distilled off under reduced pressure (T≦50° C.) until the residue was about 6 L to yield a toluenic solution of ethyl [6-aminochroman-2-yl]acetate.

¹H-NMR (400 MHz, CDCl₃) 6.61 (d, J=8.9 Hz, 1H), 6.46 (dd, J=8.9 Hz, 2.5 Hz, 1H), 6.40 (d, J=2.5 Hz, 1H), 4.37 (qd, J=7.5 Hz, 1.2 Hz, 1H), 4.18 (q, J=7.2 Hz, 2H), 3.22 (s, 2H), 2.81 (ddd, J=16.5 Hz, 5.2 Hz, 4.1 Hz, 1H), 2.58 (dd, J=15.4 Hz, 7.4 Hz, 1H), 2.02 (dm, J=13.5 Hz, 1H), 1.75 (m, 1H), 1.07 (t, J=7.2 Hz, 3H).

¹³C-NMR (100 MHz, CDCl₃) 107.9, 147.5, 139.4, 122.1, 117.3, 115.9, 115.0, 72.1, 60.6, 40.6, 27.3, 24.5, 14.2.

In view of the above description it is believed that one of ordinary skill can practice the invention. The examples given above are non-limiting in that one of ordinary skill in view of the above will readily envision other obvious permutations and variations without departing from the principal concepts embodied therein. Such permutations and variations are also within the scope of the disclosure.

What is claimed is:

1. A process for making a compound according to the formula 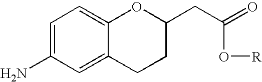
wherein R is H or an alkyl group,
comprising:
(a) reacting 6-nitrocoumarin with at least one hydrogenation agent to hydrogenate the 6-nitro group to a 6-amino group and concurrently or in a further step hydrogenate the 3-4 alkene bond on the lactone ring to produce a R¹-6-amino-2-chromanone compound as follows: 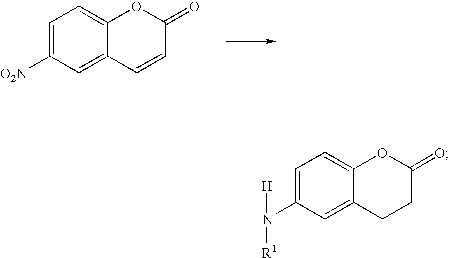
wherein the R¹ group is a hydrogen atom or a removable amino protecting group;
(b) reducing the 2 position carbonyl group of the protected 6-amino-2-chromanone from (a) to form a protected 6-amino-2-hydroxychromane as follows: 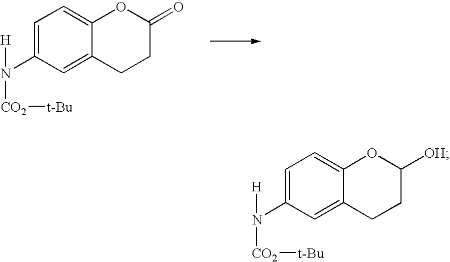
(c) condensing the hydroxychromane compound of (b) with a triphenylphosphorane compound in the presence of a suitable base at a temperature of about 50-100° C., to afford the acetate compound as follows: 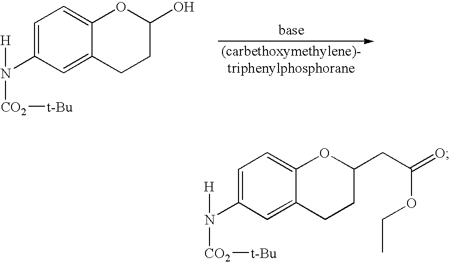
(d) removing the protecting group from the 6-amino group, as follows:

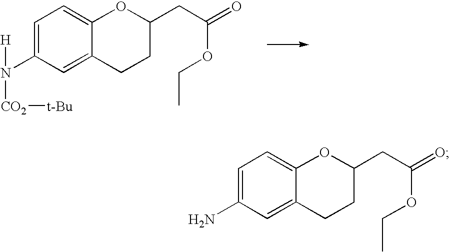

2. The process according to claim 1, further comprising forming the free acetic acid side chain or performing a transesterification process step to provide a compound of the formula: 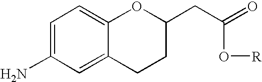
wherein R is H or an alkyl group.

3. The process according to claim 1, further comprising forming a halide salt of the 6-amino group and isolating the salt as a polymorphic or crystalline material.

4. The process according to claim 1, for making a compound according to the formula: 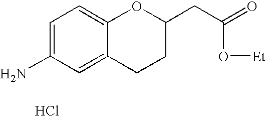
comprising:
(a) reacting 6-nitro coumarin with hydrogen in the presence of palladium on carbon to reduce the 6-nitro group to a 6-amino group and hydrogenating the 3-4 alkene bond at a temperature between about 20° C. and 45° C. and a pressure of about 2 bar G until the end of exothermicity and then raising the temperature to about 50° C. to about 60° C. at a pressure of about 4 bar for about 5 hours, wherein the hydrogenation is conducted in THF as a solvent and in the presence of di-tert-butoxydicarbonate to protect the amino group and produce 6-(tert-butoxycarbonylamino)-2-chromanone compound as follows: 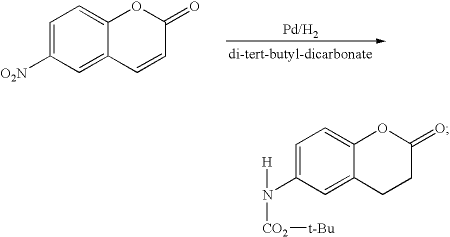
(b) reducing the carbonyl group (2-oxo group) of the protected chromanone compound of (a) to a 2-hydroxychromane compound by utilizing a DIBAL-H reduction process in the presence of an organic solvent system, at a temperature of from about −50° C. to about −65° C. like as follows: 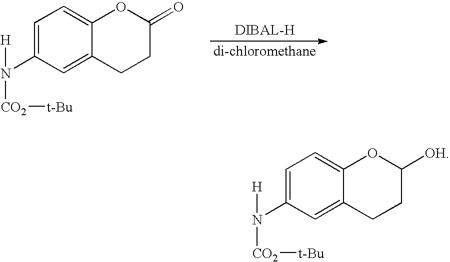
(c) reacting the 2-hydroxychromane with (carbethoxymethylene)-triphenylphosphorane in the presence of a base to append the ethyl acetate group to the ring structure in an acceptable solvent at about 50-100° C., as follows: 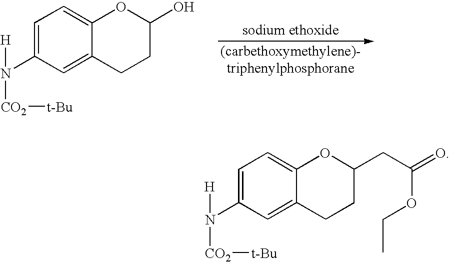
(d) removing the protecting group on the amine group by acidifying the reaction mixture with trifluoroacetic acid at about 40-80° C. to yield the free amine as follows: 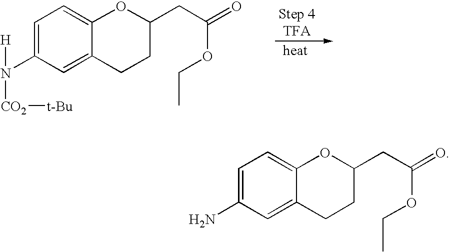
(e) reacting the free amine with a halide to form the halide salt of the amine.
